(19) United States
(12) Patent Application Publication
(10) Pub. No.: US 2008/0249061 A1
(43) Pub. Date: Oct. 9, 2008

(54) Photostable Pharmaceutical Composition Containing Brivudine for the Treatment of Herpetic Keratitis

(76) Inventors: Marc Wihsmann, Schulzendorf (DE); Reinhard Schmitz, Berlin (DE)

Correspondence Address: ARENT FOX LLP, 1050 CONNECTICUT AVENUE, N.W., SUITE 400, WASHINGTON, DC, 20036, US

(21) Appl. No.: 12/088,397

(22) PCT Filed: Sep. 27, 2006
(86) PCT No.: PCT/EP2006/009383

§ 371 Date: Mar. 27, 2008

(30) Foreign Application Priority Data
Sep. 29, 2005 (DE) 10 2005 046 769.5

(51) Int. Cl. A61K 31/7072; A61P 31/22

(52) U.S. Cl. 514/50

(57) ABSTRACT

Ophthalmic composition containing brivudine as active ingredient and a film-forming agent selected from the group consisting of polyvinyl pyrrolidone (PVP), polyvinyl alcohol (PVA) and polyacrylate (PA).

### FIELD OF THE INVENTION

This invention relates to pharmaceutical compositions containing the antiviral brivudine, which are useful for the treatment of ocular inflammatory conditions, particularly stromal keratitis induced by Herpes Simplex Virus Type 1 (HSV-1) or by Varicella-Zoster Herpes Virus (VZV). The compositions allow photo protection of the antiviral with excellent tolerability and long-lasting delivery of brivudine.

### BACKGROUND OF THE INVENTION

Inflammatory diseases of the eye can be initiated by viral, bacterial, fungal, or parasitic infection and by autoimmunity. One particularly damaging kind of ocular inflammatory condition is the keratitis induced by HSV-1 or less commonly by HSV type 2. Corneal inflammation due to HSV is particularly severe and can persist for years even when treated. HSV keratitis can confine to the outer epithelial layer of the cornea (epithelial keratitis) causing corneal ulcers with the potential to penetrate into deeper structures of the cornea (stromal keratitis) which may lead to corneal necrosis (necrotising stromal keratitis). A common sequela is corneal vascularization and scarring. Ocular complications of herpes zoster ophthalmicus, caused by Varicella Zoster Virus (VZV), may comprise non-specific conjunctivitis, dendritiform epithelial or stromal keratitis, anterior keratouveitis, retinal hemorrhage or retinitis and may also affect the optic nerve.

Currently, HSV induced epithelial keratitis is treated with antivirals while HSV stromal keratitis is treated by topically applied corticosteroids with an antiviral cover (See, e.g., Wilhelmus, K. R. et al., Opthalmology. 101:1883, 1994). However, corticosteroid therapy may prolong and possibly worsen the disease as well as introduce other effects such as enhancement of viral replication, cataracts, glaucoma, corneal melting, secondary infection, and corticosteroid dependence. (See Liesegang, T. J., Mayo Clin. Proc. 63:1092, 1988).

Pharmaceutical compositions containing an antiviral agent such as acyclovir, for ocular topical treatments, are described in several patents:

CN1342651 (CAplus AN: 2003:394238) describes the preparation, among others, of eye drop containing ganciclovir sodium dihydrate.

RU2158591 (CAplus, AN: 2002:13230) deals with the treatment of herpetic eye diseases, in particular, herpetic keratitis, with acyclovir 0.1 g in 10 ml soln. of lacrisin.

In WO2000004884 topical treatment of herpetic keratitis was achieved with a lotion containing acyclovir and adenosine.

JP10287552 (Caplus, AN 1998:693384) describes eye drop with an acyclovir suspension.

WO96/24367 relates to a pharmaceutical composition for the treatment of herpes simplex virus infections, comprising a quaternary ammonium compound as a first component.

An antiviral agent which appears to be very promising, even in the treatment of herpetic keratitis, is brivudine. (E)-5-(2-bromovinyl)-2′-deoxyuridine. Its in vitro efficacy against VZV is superior to both acyclovir and penciclovir [mean IC₅₀ in 13 clinical VZV strains: 0.001 μg/ml (brivudine), 0.2 μg/ml (acyclovir), and 0.91 μg/ml (penciclovir)] (Andrei G., Snoeck R., Reymen D. Eur. J. Clin. Microbiol. Infect.; 14; 318-319; 1995).

Furthermore, brivudine is a potent inhibitor of HSV-1 replication with a superior in vitro efficacy compared to acyclovir and penciclovir: in a panel of 23 clinical HSV-1 strains, brivudine proved to be almost twice as effective as acyclovir [mean IC₅₀: 0.52 μg/ml (brivudine) vs. 0.92 μg/ml (acyclovir)] (Andrei G., Snoeck R., Goubau P. Eur. J. Clin. Microbiol. Infect.; 11; 143-151; 1992). In two similar assays in a panel of 20 clinical isolates penciclovir showed a mean IC₅₀ of 0.6 μg/ml and 0.8 μg/ml (Weinberg A., Bate B. J., Masters H. B. Antimicrob. Agents Chemother.; 36; 2037-2038; 1992).

Earlier investigations predominantly in HSV-1 laboratory strains demonstrate an even lower IC₅₀ of brivudine between 0.004 μg/ml and 0.2 μg/ml (depending on the viral strain and the cell system) (De Clercq E., Descamps J., Verhelst G., J. Infect. Dis.; 141; 563-574; 1980; De Clercq E., Antimicrob. Agents Chemother.; 21; 661-663; 1982).

The use of brivudine for the treatment of ocular viral diseases was described in several animal and clinical tests: e.g. Antiviral Research (1984 October) 4(5) 281-291; Current eye Research. 1991 10 suppl, 193-9, but no commercial pharmaceutical composition for topical ocular use has so far appeared on the market, suggesting that some problems have still to be solved.

Brivudine, in fact, has revealed particularly unstable in the conditions normally used for topical preparations. In those conditions brivudine was found to undergo an extensive photodegradation, which reduces the concentration of the active principle, decreasing the ultimate effect of the treatment and determining a significant local irritation that can discourage from using the pharmaceutical composition.

Since promising drugs for the treatment of herpetic keratitis are not yet available (exception is acyclovir, but it is toxic in the epithelium) short term brivudine formulations are used in selected hospitals (see Krankenhauspharmazie 2002, 23, 174), but also these brivudine formulations are characterised by a rapid lost of activity due to the degradation of the active.

In WO02/056913 topical pharmaceutical formulations (not for ocular uses) containing brivudine have been described: the antiviral agent was here stabilised with a high percentage of a metal oxide pigment, preferably TiO₂.

When pigments are used in a pharmaceutical formulations they are normally part of a water in oil (W/O) resp. oil in water (O/W) suspension or—exceptionally—of a slurry; in these systems the pigments are still solid.

Since photo protection of brivudine needs amounts of pigment not less than 20% the viscosity of the formulation is very high. Considering that the sickened eye is much more sensitive towards outer influences, an ophthalmic use of this kind of formulation would generate aches and pains.

In a quite similar way, WO03051375 describes other topical formulations where the active compound, brivudine, was stabilized against photo degradation with an UV-filter, in particular with one selected in the group of derivatives of o-hydroxy-benzophenone.

The invented solutions are not suitable for ocular formulation, since chemical UV-filters are dissolved within the formulation. In this form this substances are to be expected as eye-irritant. The sickened eye is markedly more sensitive and a remarkable protecting effect of the sunscreens is to be observed at concentrations higher than 5%. Therefore the use of dissolved sunscreens should be avoided.

### DESCRIPTION OF THE INVENTION

It has been surprisingly found that ophthalmic composition containing film-forming agents selected from polyvinyl pyrrolidones (PVP), polyvinyl alcohol (PVA) and polyacrylates (PA) prevent brivudine photodegradation allowing at the same time excellent tolerability and long-lasting local delivery of the drug.

In particular, it was observed that the film forming agents according to the invention ensure recovery rates of more than 85% brivudine (see Table 1), after irradiation with 8.9 J/cm², in contrast to other thickeners, including alginats, xanthane gum, carrageenan, acacia gum, guar gum, cellulose derivatives and agar gel (best observed result was 70% recovery of brivudine after irradiation).

Formulations based on film formers are commonly used as artificial tear drops. They are approved since decades without any general or special adverse effects published. In additional studies performed by us to investigate the influence of brivudine on the drug product-tolerability, no deterioration of the pharmaceutical properties was observed. These studies also showed that brivudine remains for more than 2 hours in the ocular region without systemic absorption in the blood.

The invention therefore solves the problem of providing a brivudine-based ophthalmic product characterized by a low content of active substance and a noticeably increased stability against light-induced degradation in comparison with known formulations (without stabilising agent). In addition to the active substance, the composition may contain excipients, adjuvants, carriers, solubilisers and especially preservatives.

According to a preferred embodiment, the ophthalmic pharmaceutical formulation contains.

0.05 to 2% by weight of brivudine, 0.5 to 10% by weight of film forming agents, 0 to 2.0% by weight of preservative and a residual content of up to 100% by weight of pharmaceutically acceptable excipients, adjuvants, carriers, solubilisers.

In a particularly preferred embodiment of the invention, the ophthalmic formulation contains, besides the active principle, a film forming agent selected from the group consisting of PVP, PVA and polyacrylates, a preservative selected from the group consisting of chlorhexidine, chlorbutanol and boric acid, an antioxidant selected from the group consisting of butylated hydroxytoluene (BHT), esters of gallic acid, esters of ascorbic acid and esters of α-tocopherole. Other suitable excipients include flavours, buffers, surfactants and co-solvents.

The brivudine content may range from 0.05 to 2%, preferably from 0.05 to 1%, more preferably from 0.05 to 0.3% by the weight of the composition.

The amount of film former is preferably from 0.5 to 10%, more preferably from 1 to 5% by weight of the composition.

The amount of preservatives—if present—is preferably up to 2%, more preferably from 0.01 to 1.5% by weight of the composition.

The amount of antioxidants—if present—is preferably from 0.001 to 1%, more preferably from 0.001 to 0.1% by weight of the composition.

The formulations of this invention may contain further carriers/enhancers and adjuvants such as water, monovalent alcohols such as ethanol, colorants, thickeners, plasticizers, surfactants, polyols, esters, electrolytes, gel forming agents, polar and non-polar oils, polymers, copolymers, emulsifiers, emulsion stabilisers.

In addition, liposomes can be used to improve the delivery of the active principle.

Further adjuvants and active substances may be present, including for example vitamin A or vitamin-A derivatives; vitamin E or vitamin-E derivatives; complex vitamins, coloured or uncoloured plant extracts.

The preparation of the invention can be in the form of drops, solutions, gels or ointments, packaged in single dose or multiple dose containers.

A typical ocular preparation according to the present invention contains (% w/w):

0.1-0.3% of brivudine

1-7% of a film forming agent

0-2% of one or more preserving agents

0-2% buffer

0-0.9% NaCl

0-4% 0.1 n NaOH (up to pH ranging from 5.5 to 6.5)

0-25% surfactant

0-0.003% antioxidant

0.01-0.05% Sodium-EDTA

ad 100% water

The manufacturing is performed in an aseptic area under sterile conditions. The formulation is prepared using well known methods (mixing, stirring, homogenising, filtering) and with well known instruments (homogenisers e.g. Ultra Turrax, Silverson mixer or similar devices, sterile filters)—see, e.g., Remington's Pharmaceutical Sciences, and U.S. Pharmacopeia and Voigt, Rudolf, Pharmazeutische Technologie für Studium und Beruf, 7th revised edition, Berlin (1993).

Ophthalmic formulations containing Brivudine can be used to treat several ocular pathologies, in particular HSV-epithelial keratitis, HSV-stromal keratitis, ocular complications of herpes zoster ophthalmicus.

The effective amount of active ingredient may vary depending on factors such as the condition to be treated, the progression of the disease, the overall health conditions of the patient, the form, route and dose of administration and the severity of side effects. As used herein, the phrase “therapeutically effective dose” means a dose sufficient to ameliorate a symptom or sign of ocular inflammation. The appropriate doses can be easily determined using methods known in the art.

In appropriate circumstances, the composition according to the invention can be used for combined therapy. Preferably, the combined preparation will contain brivudine and an active ingredient selected from steroids, particularly prednisolone acetate and fluorometholone, and non-steroidal antinflammatory agents such as flurbiprofen, for simultaneous, sequential or separate administration.

The invention is further illustrated by the following non-limiting examples.

### EXAMPLES

Unless otherwise specified, the classes or groups of compounds mentioned throughout the patent description and the examples should be intended as follows:

Polyvinyl pyrrolidones: compounds known under the INCI names 1-Ethenyl-2-pyrrolidinone homopolymer and PVP and the marked name Kollidon (BASF), e.g. Kollidon 12 (molecular weight appr. 2,000-3,000), Kollidon 17 (molecular weight appr. 7,000-11,000), Kollidon 25 (molecular weight appr. 28,000-34,000) and (Kollidon 30 (molecular weight appr. 44,000-54,000), Kollidon 90 (molecular weight appr. 1,000,000-1,500,000); all qualities correspond to pharmaceutical Pharmacopoeias, see Ph. Eur. 01/2005:0685.

Polyvinyl alcohols: compounds known under the INCI names Ethenol homopolymer, PVA and the market name e.g. Kollicoat® Protect (BASF); e.g. PVA 72,000 or PVA 145,000; all used qualities correspond to pharmaceutical Pharmacopoeias, see Ph. Eur. 01/2005:1961.

Polyacrylates: compounds known under INCI names polyacrylic acid and the market name e.g. Carbopol 934P, 941, 971, 940 and 980 or Carbopol ETD 2001, 2020 and 2050, resp. Noveon AA-1; qualities used correspond to pharmaceutical Pharmacopoeias, Ph. Eur. (see 01/2005:1299).

Poloxamers: synthetic block copolymers of ethylene oxide and propylene oxide. In principal all poloxamers described in European Pharmacopoeia (P 124, 188, 237, 338 and 407) can be used. The following types were found to be usable in particular: Poloxamer 407 (average molecule mass: 9840-14600) and Poloxamer 188 (average molecule mass: 7680-9510), see Ph. Eur. 01/2005:1464.

### Example 1

The following eye drops formulation was prepared under aseptic conditions by mixing the ingredients at a temperature of 20° C. for two hours, and then adding the active first and subsequently the preservative under moderate mixing. Finally the solution is filtered aseptically through a 0.2 μm filter. Primary packaging are e.g. sterile bottles for multiple dosage:

| | |
|---|---|
| Brivudine | 0.1% |
| Kollidon 25 | 2% |
| Boric acid | 1.5% |
| Chlorhexidin acetate | 0.03% |
| 0.1n NaOH | up to pH = 6.5 |
| Sodium-EDTA | 0.01% |
| Water q.s. | ad 100% |

### Example 2

### The Eye Drops Formulation was Prepared According to a Procedure Similar to Example 1

| | |
|---|---|
| Brivudine | 0.1% |
| Povidone K 25 | 5% |
| NaCl | 0.7% |
| Chlorbutanol | 0.5% |
| 0.1n NaOH | up to pH = 5.5 |
| Sodium-EDTA | 0.01% |
| Propyl gallate | 0.002% |
| Water q.s. | ad 100% |

### Example 3

### The Eye Drops Formulation was Prepared According to a Procedure Similar to Example 1

| | |
|---|---|
| Brivudine | 0.1% |
| Polyvinyl alcohol 72000 | 0.3% |
| Boric acid | 1.8% |
| Chlorhexidin acetate | 0.02% |
| Chlorbutanol | 0.3% |
| 0.1n NaOH | up to pH = 5.5 |
| Sodium-EDTA | 0.01% |
| Butylated hydroxytoluene | 0.001% |
| Water q.s. | ad 100% |

### Example 4

### The Eye Drops Formulation was Prepared According to a Procedure Similar to Example 1

| | |
|---|---|
| Brivudine | 0.1% |
| Carbomer 980 | 6% |
| Boric acid | 1.8% |
| Chlorhexidin acetate | 0.02% |
| Chlorbutanol | 0.3% |
| 0.1n NaOH | up to pH = 5.5 |
| Sodium-EDTA | 0.01% |
| Water q.s. | ad 100% |

### Example 5

### The Eye Drops Formulation was Prepared According to a Procedure Similar to Example 1; the Primary Packaging in this Case (not Preserved Formulation) are Single Dose Containers

| | |
|---|---|
| Brivudine | 0.1% |
| Povidone K 25 | 1.4% |
| Na₂HPO₄ | 0.65% |
| NaH₂PO₄ | 0.18% |
| NaCl | 0.23% |
| Sodium-EDTA | 0.01% |
| Water q.s. | ad 100% |

### Example 6

### The Eye Drops Formulation was Prepared According to a Procedure Similar to Example 1

| | |
|---|---|
| Brivudine | 0.3% |
| Povidone K 25 | 2% |
| Poloxamer 407 | 10% |
| Poloxamer 188 | 15% |
| Boric acid | 1.5% |
| Chlorhexidin acetate | 0.03% |
| 0.1n NaOH | up to pH = 6.5 |
| Sodium-EDTA | 0.01% |
| Water q.s. | ad 100% |

### Example 7

### Photostability Test

TABLE 1
Exemplary recovery rate of brivudine in film former (PVP)
containing solution (25 Wh/m² = 8.9 J/cm² (UVA/VIS) ≅ 67 min)

recovered brivudine [%]

| | | formulation |
|---|---|---|
| Irradiation | Formulation without | corresponding to |
| time (min) | film former | example 1 |
| 0 | 99.8 | 101.5 |
| 10 | 96.7 | 99.8 |
| 20 | 87.9 | 98 |
| 30 | 77.4 | 93.5 |
| 67 | 33.4 | 85.5 |

Table 1 shows the brivudine amount in formulations with and without film forming agent depending on the irradiation time. Here can be observed that after irradiation of one hour only 33.4% of the starting amount of brivudine is recovered if no film former is contained.

However, it can be seen that the film forming agent causes a significant stabilisation against light induced degradation.

The photo stability tests themselves have been performed using a SUNTEST CPS+ instrument of the company Atlas. The applied irradiation energy is indicated below:
Irradiation energy (time): 1.3 J/cm² (10 min), 2.6 J/cm² (20 min), 4.0 J/cm² (30 min), 8.9 J/cm² (67 min), 26.7 J/cm² (201 min)
Wave length range: 320-800 nm

Table 2 reports a direct comparison between some representative formulations containing 2% film forming agent and a formulation without film former. Just after approx. one hour the protected formulation contains 50% more brivudine than the unprotected drug.

In Table 3 the recovery of brivudine in formulation containing alternative film-forming agents is shown: These film-forming agents did not appear as satisfactory solution for the present invention.

Finally Table 4 reports the results of an intra ocular bioavailability study in rabbit. The presence of the active substance in the cornea was observed even after 6 hours. In contrast, the active was never found in blood.

The results of this study indicate that the formulation should be applied only 4 to 5 times daily and that the active is not absorbed systemically.

TABLE 2
Comparison of the brivudine photo stability in formulations
with and without film forming agent (PVP)

recovered brivudine [%]

| | | formulation | | | |
|---|---|---|---|---|---|
| | formulation | corresponding | formulation | formulation | |
| | corresponding | to example 2 | corresponding | corresponding | |
| | to example 1 | (PVA, | to example 6 | to example 3 | formulation |
| irradiation | (PVP, | Chlorbutanol, | (PVP, without | (PA, | containing |
| energy | Chlorhexidin) | Chlorhexidin) | preservative) | Chlorhexidin) | no film |
| [J/cm²] | [%] | [%] | [%] | [%] | former [%] |
| 0 [J/cm²] | 101.5 | 100.2 | 100.4 | 99 | 99.8 |
| 8.9 [J/cm²] - | 85.5 | 83.8 | 87.3 | 83.3 | 33.4 |
| 67 min | | | | | |
| irradiation | | | | | |

TABLE 3
Comparison of the brivudine photo stability in formulations
containing alternative film forming agents

| | recovered brivudine [%] | | | |
|---|---|---|---|---|
| | formulation | formulation | formulation | |
| irradiation | containing agar | containing | containing | formulation |
| energy | gel as | hydroxyl | sodium | containing |
| [J/cm²] | thickener | propylcellulose | alginate | carrageenan |
| 0 | 100.8 | 100.3 | 102.4 | 101.1 |
| [J/cm²] | | | | |
| 8.9 | 35.7 | 70.7 | 33.8 | 24.0 |
| [J/cm²] - | | | | |
| 67 min | | | | |
| irradiation | | | | |

Biological Test

TABLE 4
Brivudine (BVDU) mean concentration in the cornea, in the
aqueous humour and in blood plasma after application of formulation
corresponding to example 5

| | | | BVDU in | |
|---|---|---|---|---|
| | | BVDU | rabbit | BVDU |
| | | in rabbit | aqueous | in rabbit |
| Number of | | cornea | humour | blood plasma |
| Animals | Time | [ng/cornea] | [ng/ml] | [ng/0.5 ml] |
| 4 | 15 min | 36.15 | 23.35 | 0 |
| 4 | 30 min | 21.1 | 30.97 | 0 |
| 4 | 60 min | 57.57 | 59.55 | 0 |
| 4 | 2 h | 11.99 | 19.62 | 0 |
| 4 | 4 h | 3.81 | 5.6 | 0 |
| 4 | 6 h | 2.69 | 0 | 0 |

1-17. (canceled)

18. Ophthalmic composition containing from 0.05 to 0.3% w/w of brivudine as active ingredient and a film-forming agent selected from the group consisting of polyvinyl pyrrolidone (PVP), polyvinyl alcohol (PVA) and polyacrylate (PA).

19. A composition according to claim 18, in which the amount of the film-forming agent ranges from 0.5 to 10% w/w.

20. A composition according to claim 19, in which said amount ranges from 1 to 5% w/w.

21. A composition according to claim 18, further containing a preservative agent in amounts up to 2% w/w.

22. A composition according to claim 21, in which said amount ranges from 0.01 to 1.5% w/w.

23. A composition according to claim 18, further containing an antioxidant agent in amounts from 0.001 to 12% by weight.

24. A composition according to claim 23, in which said amount is from 0.001 to 0.1% w/w.

25. A composition according to claim 18, which is in form of eye-drops, solution, gel, ointment.

26. A composition according to claim 25, which is packaged in single dose or multiple dose containers.

27. A composition according claim 18, containing 0.1-0.3% w/w brivudine; 1-7% w/w film-forming agent; 0-2% w/w preserving agents; 0-2% buffer; 0-0.9% NaCl; 0-4% 0.1N NaOH to give a pH ranging from 5.5 to 6.5; 0-25% surfactant; 0-0.003% antioxidant; 0.01-0.05% Sodium-EDTA; ad 100% water.

28. A combined preparation containing an ophthalmic composition in accordance with claim 18 and a steroidal or non-steroidal antiinflammatory drug for simultaneous, sequential or separate use in the treatment of ocular diseases.

29. A combined preparation according to claim 28, wherein the steroid is prednisolone acetate or fluorometholone and the non-steroidal antinflammatory drug is flurbiprofen.

30. The use of an ophthalmic composition according to claim 18 for the preparation of a medicament for the treatment of HSV-epithelial keratitis, HSV-stromal keratitis, ocular complications of herpes zoster ophthalmicus.

31. The use of a film-forming agent selected from the group consisting of polyvinyl pyrrolidones (PVP), polyvinyl alcohol (PVA), polyacrylates (PA), for the preparation of an ophthalmic composition containing brivudine as active principle.
